(19) United States
(12) Patent Application Publication
(10) Pub. No.: US 2026/0345036 A1
(43) Pub. Date: Nov. 5, 2026

(54) SYSTEM AND METHOD FOR GLUCOSE ESTIMATION

(71) Applicant: Laxmi Therapeutic Devices, Inc., Goleta, CA (US)

(72) Inventors: Jason GOLDBERG, Sherman Oaks, CA (US); William Peter VAN ANTWERP, Santa Clarita, CA (US)

(21) Appl. No.: 19/655,690

(22) Filed: Apr. 22, 2026

Related U.S. Application Data
(60) Provisional application No. 63/797,786, filed on Apr. 30, 2025.

(51) Int. Cl. A61B 5/145; A61B 5/00; A61B 5/1473

(52) U.S. Cl. CPC A61B 5/14532; A61B 5/14735; A61B 5/6846; A61B 5/725; A61B 5/7271

(57) ABSTRACT

A system and method for glucose estimation. In some embodiments, a system includes: a first glucose-sensing electrode; and a second glucose-sensing electrode, the first glucose-sensing electrode and the second glucose-sensing electrode being configured to be implanted, separated by less than 5 mm, in a subject.

### CROSS-REFERENCE TO RELATED APPLICATION(S)

The present application claims priority to and the benefit of U.S. Provisional Application No. 63/797,786, filed Apr. 30, 2025, entitled “Non-Linear Analyte Sensing with Additional Correction”, the entire content of which is incorporated herein by reference.

### FIELD

One or more aspects of embodiments according to the present disclosure relate to glucose measurement, and more particularly to a continuous glucose monitor.

### BACKGROUND

A continuous glucose monitor may include an implantable electrode on the surface of which may occur, in operation, electrochemical reactions that may generate a current the magnitude of which depends on a glucose concentration in the tissue in which the electrode is implanted.

It is with respect to this general technical environment that aspects of the present disclosure are related.

### SUMMARY

According to an embodiment of the present disclosure, there is provided a system, including: a first glucose-sensing electrode; and a second glucose-sensing electrode, the first glucose-sensing electrode and the second glucose-sensing electrode being configured to be implanted, separated by less than 5 mm, in a subject.

In some embodiments, the system further includes an electrode carrier, wherein the first glucose-sensing electrode is on the electrode carrier and the second glucose-sensing electrode is on the electrode carrier.

In some embodiments, the first glucose-sensing electrode is separated from the second glucose-sensing electrode by less than 2 mm.

In some embodiments: the first glucose-sensing electrode includes a first permeable glucose barrier having a first permeability to glucose, and the second glucose-sensing electrode includes (i) a second permeable glucose barrier having a second permeability to glucose, greater than the first permeability, or (ii) no glucose barrier.

In some embodiments, the second glucose-sensing electrode includes a second permeable glucose barrier and the second permeability is at least 1.1 times the first permeability.

In some embodiments, the system further includes a processing circuit configured to calculate an estimated glucose concentration, the calculation being based on a measured current flowing through the first glucose-sensing electrode and on a measured current flowing through the second glucose-sensing electrode.

In some embodiments, the calculation is further based on a maximum reaction rate of the first glucose-sensing electrode and a maximum reaction rate of the second glucose-sensing electrode.

In some embodiments, the calculation is further based on a Michaelis constant of the first glucose-sensing electrode and a Michaelis constant of the second glucose-sensing electrode.

In some embodiments, the estimated glucose concentration is monotonic function of a true glucose concentration.

In some embodiments, the calculating produces an estimated glucose concentration that varies by no more than 5% with a change of 10% in a gain factor common to the first glucose-sensing electrode and the second glucose-sensing electrode.

In some embodiments: the calculating includes evaluating a function, the function mapping a first input value x and a second input value y to an output value z, the function having the property that for x=k y, with k being a constant, and for x ranging from a first value x0 to a second value x1=1.5 x0, z changes by less than 10%.

In some embodiments, the calculating includes evaluating a function, the function mapping a first input value x and a second input value y to an output value z, the function having the property that for x=ky, for x ranging from a first value x0 to a second value x1=1.5 x0, and for k ranging from a value of 0.7 to a value of 1.3, z is within 10% of (k A−B)/(1−C k) wherein A, B, and C are constants.

In some embodiments, A/(BC) is within 10% of a Michaelis constant of the first glucose-sensing electrode divided by a Michaelis constant of the second glucose-sensing electrode.

In some embodiments, C is within 10% of a maximum reaction rate of the first glucose-sensing electrode divided by a maximum reaction rate of the second glucose-sensing electrode.

In some embodiments, the calculating includes executing an Extended Kalman Filter.

In some embodiments, a state vector of the Extended Kalman Filter includes a glucose concentration.

In some embodiments, a state vector of the Extended Kalman Filter includes a background glucose-sensing electrode current.

In some embodiments, a state vector of the Extended Kalman Filter includes a gain ratio.

According to an embodiment of the present disclosure, there is provided a method, including: calculating an estimated glucose concentration based on a first sensor signal, from an output of a first glucose-sensing electrode, and a second sensor signal, from an output of a second glucose-sensing electrode, a fit of the current of first glucose-sensing electrode as a function of glucose concentration to a Michaelis-Menten equation resulting in a first Michaelis constant, a fit of the current of second glucose-sensing electrode as a function of glucose concentration to a Michaelis-Menten equation resulting in a second Michaelis constant, differing from the first Michaelis constant by at least 1%.

In some embodiments: the calculating including evaluating a function, the function mapping a first input value x and a second input value y to an output value z, the function having the property that for x=ky, with k being a constant, and for x ranging from a first value x0 to a second value x1=1.5 x0, z changes by less than 10%.

In some embodiments, the function further has the property that for x=k y, for x ranging from a first value x0 to a second value x1=1.5 x0, and for k ranging from a value of 0.7 to a value of 1.3, z is within 10% of (k A−B)/(1−C k) wherein A, B, and C are constants.

In some embodiments, A/(BC) is within 10% of a Michaelis constant of the first glucose-sensing electrode divided by a Michaelis constant of the second glucose-sensing electrode.

In some embodiments, C is within 10% of a maximum reaction rate of the first glucose-sensing electrode divided by a maximum reaction rate of the second glucose-sensing electrode.

In some embodiments, the calculating includes executing an Extended Kalman Filter.

In some embodiments, a state vector of the Extended Kalman Filter includes a glucose concentration.

In some embodiments, a state vector of the Extended Kalman Filter includes a background glucose-sensing electrode current.

In some embodiments, a state vector of the Extended Kalman Filter includes a gain ratio.

According to an embodiment of the present disclosure, there is provided a system, including: a first glucose-sensing electrode; and a second glucose-sensing electrode, the first glucose-sensing electrode being implanted in a first tissue space of a subject, the second glucose-sensing electrode being implanted in a second tissue space of the subject, the tissues being selected such that their respective glucose concentrations remain within 5% of each other.

### BRIEF DESCRIPTION OF THE DRAWINGS

These and other features and advantages of the present disclosure will be appreciated and understood with reference to the specification, claims, and appended drawings wherein:

FIG. 1A is a plan view of an electrode carrier and a plurality of electrodes, according to an embodiment of the present disclosure;

FIG. 1B shows a cross-sectional view of a working electrode, according to an embodiment of the present disclosure;

FIG. 1C is a block diagram of a system for sensing glucose concentrations, according to an embodiment of the present disclosure;

FIG. 1D is a graph of sensor responses, according to an embodiment of the present disclosure;

FIG. 2 is a graph of simulated noisy measurements, according to an embodiment of the present disclosure;

FIG. 3 is a graph of state vector estimation results, according to an embodiment of the present disclosure;

FIG. 4 is a graph of state vector estimation results, according to an embodiment of the present disclosure;

FIG. 5 is a graph of state vector estimation results, according to an embodiment of the present disclosure; and

FIG. 6 is a graph of state vector estimation results, according to an embodiment of the present disclosure.

### DETAILED DESCRIPTION

The detailed description set forth below in connection with the appended drawings is intended as a description of exemplary embodiments of a system and method for glucose estimation provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

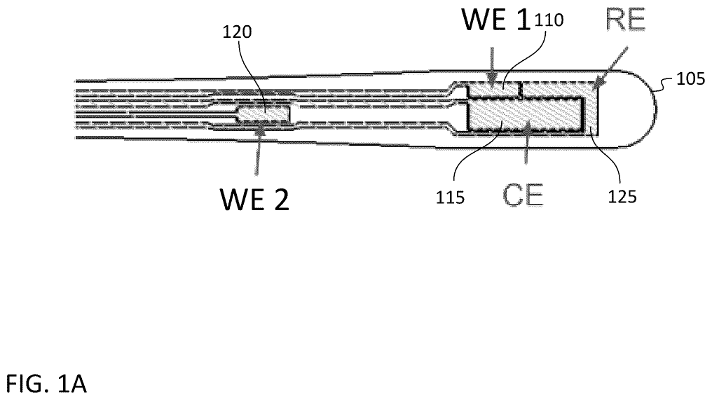

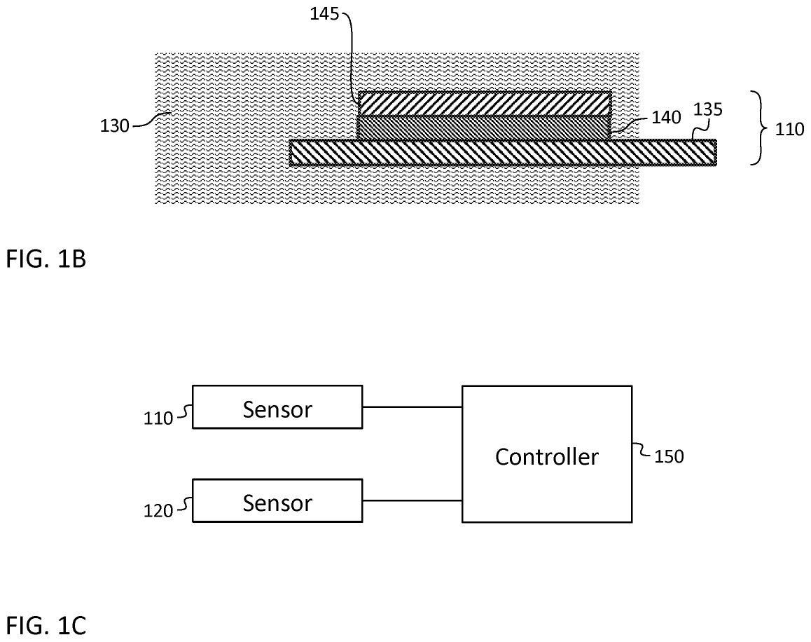

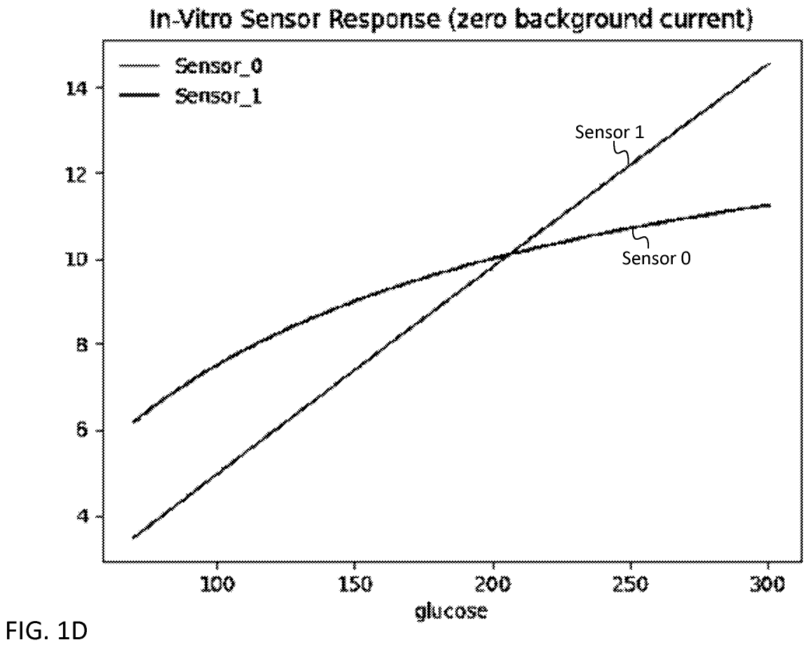

FIG. 1A shows a portion of a system for sensing glucose concentrations, e.g., a continuous glucose monitor (CGM), in some embodiments. An electrode carrier 105, which may be a thin biocompatible organic dielectric sheet, suitable for implantation into the dermis (or into the subcutaneous tissue) of a patient, or “subject”, supports one or more electrodes. Each of the electrodes may consist of (e.g., include) a thin layer of platinum, with one or more coatings, including a coating that includes glucose oxidase or a coating that operates as a permeable glucose barrier.

For example, a first working electrode 110 may have a coating containing glucose oxidase, and, on the coating, a membrane that is permeable to oxygen and partially permeable to glucose, which may operate as a permeable glucose barrier. The characteristics (e.g., the thickness or the composition) of the membrane may be selected to achieve a target permeability to glucose.

In operation, glucose and oxygen may diffuse through the membrane to reach the coating containing glucose oxidase, and the glucose oxidase may convert the glucose to hydrogen peroxide, by catalyzing the following reaction:

glucose+O₂+H₂O→gluconic acid+H₂O₂.

The hydrogen peroxide then causes, through an electrochemical reaction, a current to flow in the first working electrode 110. The return current may flow through a counter electrode 115.

If the rate of the reaction is limited by the concentration of glucose (e.g., if oxygen is abundant at the coating, or if the amount of enzyme is small), then the rate of reaction and the electrode current may be proportional to the concentration of glucose in the interstitial fluid which surrounds the electrode carrier 105 and the electrodes, and the electrode current may be a substantially linear function of the glucose concentration. If the permeability of the membrane is high, however, or if the membrane is absent, then at sufficiently high glucose concentrations, oxygen at the coating may be depleted, and the rate of reaction and the current flowing in the first working electrode 110 may be limited by the oxygen concentration, which in turn may be limited by the rate of diffusion of oxygen through the interstitial fluid. As such, for a highly permeable (or absent) membrane, the electrode current may be a nonlinear function of the glucose concentration, with the rate of change of current with glucose concentration being lower at high glucose concentration than at low glucose concentrations. The electrode current as a function of glucose concentration may be modeled using the Michaelis-Menten equation (discussed in further detail below); the sensor response (electrode current as a function of glucose concentration) is illustrated in FIG. 1D (discussed in further detail below). The extent to which the sensor response is linear may depend on the value of a parameter referred to as the “Michaelis constant”, which may depend on the permeability of the membrane, and a higher value of which may result in a more linear sensor response.

Other species (such as urea or acetaminophen) may participate in a similar electrochemical reaction to that of hydrogen peroxide and therefore generate an additional current in the first working electrode 110, which may be referred to as a background current. This background current (or simply “background”) may be a source of error in the measurement of glucose and, in some embodiments, steps (discussed in further detail below) are taken to estimate the background and subtract it from the measured electrode current before calculating the glucose concentration.

In some embodiments, it may be useful for the system for sensing glucose concentrations to include two electrodes (i) that are sufficiently close together that the glucose concentration around both is substantially the same and so that the concentration of background-generating species is also substantially the same, and (ii) one of which is more linear than (e.g., has a higher Michaelis constant than) the other. In the embodiment of FIG. 1A, this is accomplished by including both the first working electrode 110 (WE 1) and a less linear electrode, e.g., a second working electrode 120 (WE 2), which may include a coating containing glucose oxidase, and lack a membrane) on the same electrode carrier 105, separated by a small distance (e.g. by less than 3 mm). In some embodiments, the second working electrode 120 has a glucose barrier (e.g., a membrane) which is more permeable (or less permeable) than that of the first working electrode 110. A reference electrode (RE) 125, which may also be on the same electrode carrier 105 may be set to a fixed potential (e.g., −585 mV) relative to the working electrodes 110, 120.

FIG. 1B shows a cross-sectional view of the first working electrode 110, within the interstitial fluid 130. The first working electrode 110 includes a conductive layer 135 (e.g., a layer of platinum), a coating containing glucose oxidase 140, and a glucose barrier, e.g., a membrane 145 (e.g., a membrane that is permeable to oxygen and partially permeable to glucose). FIG. 1C is a block diagram of a system for sensing glucose concentrations, including one or more sensors (e.g., two sensors, one of which may be the first working electrode 110 and the other of which may be the second working electrode 120) and a controller 150. The controller 150 may include interface circuits (e.g., a bias circuit for applying a bias voltage to the first working electrode 110 and to the second working electrode 120), one or more amplifiers (e.g., transimpedance amplifiers) for generating amplified signals corresponding to the electrode currents, and one or more analog to digital converters for converting the signals to digital form. The controller may further include a processing circuit for calculating estimated glucose concentrations from the digital signals.

A continuous glucose monitor may be calibrated using one or more glucose measurements obtained from a reference source, e.g., a finger-stick-based glucose monitor. These measurements enable estimation of sensor sensitivity and, possibly, sensor background current. Some CGMs may be pre-calibrated, which may make it possible to avoid a finger-stick-based calibration measurement. Tight control of the manufacturing process may be used to produce highly consistent sensors of specified in-vivo calibration over both the shelf-life of the CGM and over the course of the actual wear. Such requirements may increase production costs, however.

As such, some embodiments include a reference-free alternative to full in-vivo calibration and to full calibration for a CGM. Such a CGM may include two or more generally nonlinear glucose sensors (e.g., working electrodes). These sensors may undergo only in-vitro calibration as part of the manufacturing process. Such an in-vitro calibration is described in U.S. patent application Ser. No. 19/355,076, filed Oct. 10, 2025, entitled “ANALYTE SENSORS WITH MODIFIED CALIBRATION”, the entire content of which is incorporated herein by reference. It is assumed in-vivo and in-vitro sensitivities are related by an unknown scale factor that is shared by all sensors in an array of sensors fabricated at the same time. Certain common forms of sensor non-linearity may be exploited to estimate in-vivo glucose without explicit, a-priori knowledge of the in-vitro-to-in-vivo sensitivity scale factor or sensor background current. The techniques may be generalized to slowly varying scale factor and background current.

In a CGM system including an array of N glucose-oxidase sensors, the in-vitro relationship, for the kth sensor, between input glucose and output sensor current at time t may be written generally as:

Iₖ(t)=aₖ[g(t)]+b k∈{0,1, . . . ,N−1} (1)
where Iₖ (t) is the output sensor current, g(t) is in-vitro glucose concentration, aₖ[·] is a generally non-linear function, and b (or β, used elsewhere herein) is the assumed common background current.

For example, the function aₖ[·] may be the nonlinear function describing Michaelis-Menten type reaction rates, which may be used to model the behavior of each of the glucose sensors:

$${a}_{k} [ g ] = {V}_{max}^{( k )} \frac{g}{g + {K}_{M}^{( k )}} \qquad ( 2 )$$

where

$${V}_{max}^{( k )} and {K}_{M}^{( k )}$$

are kth sensor's maximum reaction rate and Michaelis constant, respectively. The maximum reaction rate may depend on, for example, the size and shape of the electrode of the sensor, and on the presence and permeability of the glucose barrier.

When the Michaelis constant is much larger than a maximum expected glucose concentration gₘₐₓ, the sensor approaches linearity:

$${a}_{k} [ g ( t ) ] \approx α g ( t ) {α}_{k} = \frac{{V}_{max}^{( k )}}{{K}_{M}^{( k )}} , g ( t ) \leq {g}_{max} ≪ {K}_{M}^{( k )} \qquad ( 3 )$$

where α is the (near) linear sensor's in-vitro sensitivity.

An in-vivo sensor model may be formulated, based on certain assumptions. For simplicity, blood-to-interstitial glucose diffusion effects may be assumed negligible. For example, although such diffusion effects may be significant for subcutaneous sensing, they may be negligible for dermal sensing. As such, instantaneous blood and interstitial glucose concentrations are assumed to be very similar. Also, it may be assumed that in the absence of noise and background current, each sensor's in-vivo and in-vitro responses are related by a multiplicative scale factor f(t), which is the same for all of the sensors in the array of N sensors. In addition, a common background current β(t) is observed at the output of each sensor. Variations in f(t) and β(t) are assumed to occur slowly.

These assumptions lead to an in-vivo sensor current model of the form:

Iₖ(t)=f(t)·aₖ[g(t)]+β(t)+nₖ(t),k∈{0,1, . . . ,N−1}. (4)
where nₖ(t) is an additive noise term for the kth sensor. For Michaelis-Menten type kinetics:

$${I}_{k} ( t ) = f ( t ) \cdot {V}_{max}^{( k )} \frac{g}{g + {K}_{M}^{( k )}} + β ( t ) + {n}_{k} ( t ) k ∈ \{ 0 , 1 , … , N - 1 \} . \qquad ( 5 )$$

When a sensor response is near linear (e.g., when the Michaelis constant is much larger than a glucose concentration), the current may be given by the following equation:

Iₖ(t)=f(t)·αₖg(t)+β(t)+nₖ(t) (6)

Various random process models for the sensor noise may be considered. A simple model is that of additive white gaussian noise (AWGN) of zero mean and standard deviation σₙ. Alternatively, more complex models based on, e.g., Johnson distributed data may be used.

A simple configuration with two sensors (N=2), a first sensor and a second sensor, may be employed to illustrate how glucose can be recovered without full calibration or finger stick references.

Both sensors may undergo the same low-pass filtering operations to attenuate sensor noise. Such filtering may render nₖ(t) negligible. Lastly, for the moment, assume β(t) is known and removed from each in-vivo sensor's output. This leaves:

$${I}_{0} ( t ) \approx f ( t ) \cdot {V}_{max}^{( 0 )} \frac{g ( t )}{g ( t ) + {K}_{M}^{( 0 )}} \qquad ( 7 )$$

$${I}_{1} ( t ) \approx f ( t ) \cdot {V}_{max}^{( 1 )} \frac{g ( t )}{g ( t ) + {K}_{M}^{( 1 )}}$$

where I₀(t),

$${V}_{max}^{0} , and {K}_{M}^{( 0 )}$$

are the sensor current, the maximum reaction rate, and the Michaelis constant of the first sensor,

$${I}_{1} ( t ) , {V}_{max}^{1} , and {K}_{M}^{( 1 )}$$

are the sensor current, the maximum reaction rate, and the Michaelis constant of the second sensor, and f(t) and g(t) are unknown. Glucose can be estimated without knowing the in-vitro-to-in-vivo scale factor by forming the ratio of the two filtered sensor currents:

$$\frac{{I}_{0} ( t )}{{I}_{1} ( t )} \approx \frac{{V}_{max}^{( 0 )}}{{V}_{max}^{( 1 )}} \frac{g ( t ) + {K}_{M}^{( 1 )}}{g ( t ) + {K}_{M}^{( 0 )}} \qquad ( 8 )$$

such that f(t) is canceled. Rearranging:

$$r ( t ) ≡ \frac{{V}_{max}^{( 1 )}}{{V}_{max}^{( 0 )}} \frac{{I}_{0} ( t )}{{I}_{1} ( t )} \approx \frac{g ( t ) + {K}_{M}^{( 1 )}}{g ( t ) + {K}_{M}^{( 0 )}} \qquad ( 9 )$$

$$\to r ( t ) ( g ( t ) + {K}_{M}^{( 0 )} ) \approx g ( t ) + {K}_{M}^{( 1 )}$$

$$\to r ( t ) {K}_{M}^{( 0 )} - {K}_{M}^{( 1 )} \approx g ( t ) ( 1 - r ( t ) )$$

yields a glucose estimate of the form:

$$\hat{g} ( t ) = \frac{r ( t ) {K}_{M}^{( 0 )} - {K}_{M}^{( 1 )}}{1 - r ( l )} . \qquad ( 10 )$$

It may be noted that unless the Michaelis constants differ

$$( {K}_{M}^{( 0 )} \neq {K}_{M}^{( 1 )} )$$

the ratio of sensor currents is not a function of glucose.

The same approach may be used when one of the sensors (e.g., the first sensor) is essentially linear:

$${I}_{0} ( t ) \approx f ( t ) \cdot α g ( t ) \qquad ( 11 )$$

$${I}_{1} ( t ) \approx f ( t ) \cdot {V}_{max}^{( 1 )} \frac{g ( t )}{g ( t ) + {K}_{M}^{( 1 )}}$$

with sensor current ratio of the form:

$$\frac{{I}_{0} ( t )}{{I}_{1} ( t )} \approx \frac{α}{{V}_{max}^{( 1 )}} ( g ( t ) + {K}_{M}^{( 1 )} ) \qquad ( 12 )$$

such that:

$$r ( t ) ≡ \frac{{V}_{max}^{( 1 )}}{α} \frac{{I}_{0} ( t )}{{I}_{1} ( t )} \approx g ( t ) + {K}_{M}^{( 1 )} \qquad ( 13 )$$

which gives rise to a glucose estimate:

$$\hat{g} ( t ) = r ( t ) - {K}_{M}^{( 1 )} . \qquad ( 14 )$$

In some embodiments, the in-vitro-to-in-vivo response scale factor is estimated. This may be accomplished using any of several different possible approaches. One block-based approach uses least-squares estimation.

For the case of constant scale factor (f(t)=f) the currents measured in L measurements from each of N=2 sensors where one of the sensors is linear may be written as:

$$i = fa + n \qquad ( 15 )$$

$$i = [ \begin{matrix}\begin{matrix}\begin{matrix}\begin{matrix}\begin{matrix}\begin{matrix}\begin{matrix}{I}_{0} ( {t}_{0} ) \\ {I}_{0} ( {t}_{1} )\end{matrix} \\ ⋮\end{matrix} \\ {I}_{0} ( {t}_{L - 1} )\end{matrix} \\ {I}_{1} ( {t}_{0} )\end{matrix} \\ {I}_{1} ( {t}_{1} )\end{matrix} \\ ⋮\end{matrix} \\ {I}_{1} ( {t}_{L - 1} )\end{matrix} ] a = [ \begin{matrix}\begin{matrix}\begin{matrix}\begin{matrix}\begin{matrix}\begin{matrix}\begin{matrix}α g ( {t}_{0} ) \\ α g ( {t}_{1} )\end{matrix} \\ ⋮\end{matrix} \\ α g ( {t}_{L - 1} )\end{matrix} \\ {V}_{max}^{( 1 )} \frac{g ( {t}_{0} )}{g ( {t}_{0} ) + {K}_{M}^{( 1 )}}\end{matrix} \\ {V}_{max}^{( 1 )} \frac{g ( {t}_{1} )}{g ( {t}_{1} ) + {K}_{M}^{( 1 )}}\end{matrix} \\ ⋮\end{matrix} \\ {V}_{max}^{( 1 )} \frac{g ( {t}_{L - 1} )}{g ( {t}_{L - 1} ) + {K}_{M}^{( 1 )}}\end{matrix} ] n = [ \begin{matrix}\begin{matrix}\begin{matrix}\begin{matrix}\begin{matrix}\begin{matrix}\begin{matrix}{n}_{0} ( {t}_{0} ) \\ {n}_{0} ( {t}_{1} )\end{matrix} \\ ⋮\end{matrix} \\ {n}_{0} ( {t}_{L - 1} )\end{matrix} \\ {n}_{1} ( {t}_{0} )\end{matrix} \\ {n}_{1} ( {t}_{1} )\end{matrix} \\ ⋮\end{matrix} \\ {n}_{1} ( {t}_{L - 1} )\end{matrix} ] \qquad ( 16 )$$

The true glucose values in a may then be replaced with the glucose estimates from Equation 10 or 14 to form â, an estimate of a, such that:

i≈fâ+n. (17)

A least-squares estimate of f may then be obtained as:

$$\hat{f} = arg {min}_{\overline{f}} {‖ i - \tilde{f} \hat{a} ‖}^{2} = \frac{{\hat{a}}^{T}}{{\hat{a}}^{T} \hat{a}} i \qquad ( 18 )$$

where ∥·∥ is the Euclidean norm of a vector, and [·]^{T} denotes transpose. For slowly varying conversion factor, a simple linear model for f(t) may be used: f(t)=f₀+mt. A straightforward generalization of Equation 18 yields least squares estimates of the parameters f₀ and m defining the time variation of the scale factor.

Glucose Values

$${\{ g ( {t}_{k} ) \}}_{k = 0}^{L - 1} ,$$

scale factor f, and background current β (both Glucose values assumed constant) may be jointly estimated in terms of a nonlinear least squares optimization:

$${\{ \hat{g} ( {t}_{k} ) \}}_{k = 0}^{L - 1} , \hat{f} , \hat{β} = {argmin}_{{\{ \tilde{g} ( {t}_{k} ) \}}_{k = 0}^{L - 1} , \tilde{f} , \tilde{β}} {‖ i = \tilde{f} \tilde{a} - \tilde{β} 1 ‖}^{2} \qquad ( 19 )$$

where ã is based on

$${\{ \tilde{g} ( {t}_{k} ) \}}_{k = 0}^{L - 1} ,$$

and 1 is a column vector of ones of appropriate length.

As in the case of estimating the in-vitro-to-in-vivo response scale factor, this approach may be expanded to handle simple variations in f(t) and β(t). However, irrespective of the calibration parameters, Equation 19 is a complicated non-linear optimization problem in

$${\{ \tilde{g} ( {t}_{k} ) \}}_{k = 0}^{L - 1} .$$

Numerical methods may successfully converge to a global optimum if they are seeded with good initial guesses for glucose. (For constant scale factor and background current, f and β enter into the problem linearly and may be estimated for any given, hypothesized glucose sequence.) When β is known and removed, such guesses may be generated from a ratio-of-sensor-currents type estimator such as that of Equation 10 or 14.

In some CGMs, a separate, enzyme-inactivated electrode (or a bare electrode lacking an enzyme coating) is used to directly measure background current. Such measurements may be subtracted from the current generated at one of the working electrodes 110, 120 to cancel background current. For example, in a CGM with three or more electrodes, at least one enzyme-inactivated or bare electrode provides a direct measurement of background current. This background current may be subtracted from the currents of the working electrodes 110, 120, with the subsequent glucose estimation based on Equation 10 or 14.

In a CGM with two glucose-oxidase sensors, the sensors may generate in-vivo measurements according to the model of Equation 4, over some time interval t∈[0,T). Then, at time t=T, the enzyme may be inactivated on one of the sensors (e.g., using a high-voltage electrochemical pulse), such that it measures (a noisy version of) the background current. This background current may be subtracted from the remaining, glucose-measuring sensors. Then, glucose may be estimated as described earlier. In such an approach, real-time glucose estimation may be performed for t≥T only. (At time T, glucose over t∈[0,T) can be estimated retrospectively.) For example, data may be collected over t∈[0,T). Over this interval, glucose, f, and β may all be unknown. Once one of the sensors is inactivated, a direct measurement of β is available, which can be subtracted from the sensor data over [0, T). Then, a method like that of Equation 10 may be used to estimate f. As such, for all time, both f and β may be available, enabling straightforward estimation of glucose. This approach assumes f and β are constant. With this approach real-time estimates of glucose before time T may not be generated, but it may be possible to generate retrospective estimates of glucose before time T using the measurements of β performed after time T and using sensor currents measured before time T.

In some embodiments, a recursive or closed loop method is employed to jointly estimate glucose and the calibration parameters. For example, an Extended Kalman Filter (update equations for which are provided below) may be used.

In this context, a state vector at sample time tₖ may be defined as

$$x ( {t}_{k} ) ≡ [ \begin{matrix}g ( {t}_{k} ) \\ f ( {t}_{k} ) \\ β ( {t}_{k} )\end{matrix} ] \qquad ( 20 )$$

A constant position model may be used for all state variables, leading to the following state transition matrix:

$$F = [ \begin{matrix}1 & 0 & 0 \\ 0 & 1 & 0 \\ 0 & 0 & 1\end{matrix} ] . \qquad ( 21 )$$

For simplicity, a two sensor CGM is used in this example, yielding measurements:

$$\begin{matrix}z ( {t}_{k} ) = h ( x ( {t}_{k} ) ) + n ( {t}_{k} ) \\ = [ \begin{matrix}f ( {t}_{k} ) \cdot {V}_{max}^{( 0 )} \frac{g ( {t}_{k} )}{g ( {t}_{k} ) + {K}_{M}^{( 0 )}} \\ f ( {t}_{k} ) \cdot {V}_{max}^{( 1 )} \frac{g ( {t}_{k} )}{g ( {t}_{k} ) + {K}_{M}^{( 1 )}}\end{matrix} ] + [ \begin{matrix}β ( {t}_{k} ) \\ β ( {t}_{k} )\end{matrix} ] + [ \begin{matrix}{n}_{0} ( {t}_{k} ) \\ {n}_{1} ( {t}_{k} )\end{matrix} ]\end{matrix} \qquad ( 22 )$$

The process and measurement noise covariances are defined as:

$$Q = [ \begin{matrix}{σ}_{g}^{2} & 0 & 0 \\ 0 & {σ}_{f}^{2} & 0 \\ 0 & 0 & {σ}_{β}^{2}\end{matrix} ] R = [ \begin{matrix}{σ}_{n}^{2} & 0 \\ 0 & {σ}_{n}^{2}\end{matrix} ] \qquad ( 23 )$$

The Extended Kalman Filter locally linearizes the estimation problem via computation of the measurement Jacobian:

$$H ( {t}_{k} ) = \frac{\partial h}{\partial x} = [ \begin{matrix}f ( t ) {V}_{max}^{( 0 )} \frac{{K}_{M}^{( 0 )} )}{{( g ( {t}_{k} ) + {K}_{M}^{( 0 )} )}^{2}} & {V}_{max}^{( 0 )} \frac{g ( {t}_{k} )}{g ( {t}_{k} ) + {K}_{M}^{( 0 )}} & 1 \\ f ( t ) {V}_{max}^{( 1 )} \frac{{K}_{M}^{( 1 )} )}{{( g ( {t}_{k} ) + {K}_{M}^{( 1 )} )}^{2}} & {V}_{max}^{( 1 )} \frac{g ( {t}_{k} )}{g ( {t}_{k} ) + {K}_{M}^{( 1 )}} & 1\end{matrix} ] \qquad ( 24 )$$

In one example used for a simulation, the glucose concentration follows an offset sinusoidal glucose profile with four-hour period, given by the following equation:

$$g ( {t}_{k} ) = 50 sin ( 2 π \frac{1}{240 \times 60} {t}_{k} ) + 120 , \qquad ( 25 )$$

and a 30 second sample interval is used. Constant scale factor and background current are selected: f(t)=0.934, β(t)=1. The sensor parameters are:

$${V}_{max}^{( 0 )} = 15 {V}_{max}^{( 1 )} = 500 {K}_{M}^{( 0 )} = 100 {K}_{M}^{( 1 )} = 10 0. \qquad ( 26 )$$

The associated in-vitro sensor responses are shown in FIG. 1D where it may be seen that sensor 1 is more linear due to the large Michaelis constant of sensor 1. A combination of a linear and nonlinear sensor may be used in part because, as mentioned above, if the Michaelis constants of the sensors are the same, the ratio of the sensor currents is not a function of glucose. Moreover, the linear sensor may provide better estimates at high glucose levels since, unlike the nonlinear sensor, its response does not level off at high glucose concentration. To achieve response curves spanning similar ranges of currents over the range of glucose concentrations for which the sensor is designed, the second working electrode 120 may be made smaller (e.g., by a factor between 2 and 20) than the first working electrode 110.

In one example, the following process noise covariance parameters may be assumed:

$${σ}_{g}^{2} = 3 , {σ}_{f}^{2} = 1 {0}^{- 6} , {σ}_{β}^{2} = 2 \times {10}^{- 6} \qquad ( 27 )$$

as well as measurements corrupted by AWGN of standard deviation σₙ=0.2.

The initial state estimate vector is:

$$\hat{x} ( {t}_{0} ) = [ \begin{matrix}g ( {t}_{0} ) + 30 \\ f ( {t}_{0} ) + 0.05 \\ β ( {t}_{0} ) + 0.1\end{matrix} ] \qquad ( 28 )$$

with covariance:

$$P ( {t}_{0} \vert {t}_{0} ) = [ \begin{matrix}20 & 0 & 0 \\ 0 & 2 \times {10}^{- 4} & 0 \\ 0 & 0 & 5\end{matrix} ] \qquad ( 29 )$$

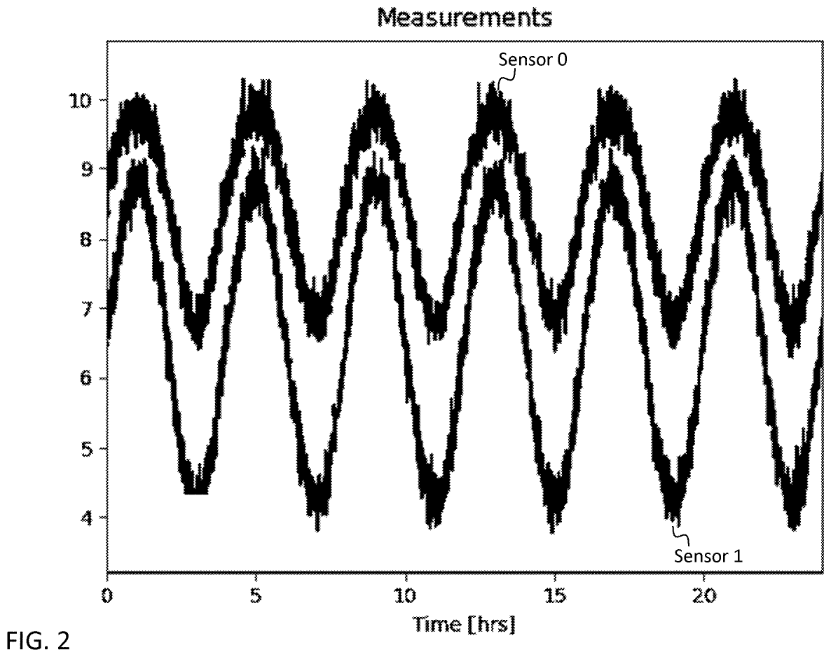

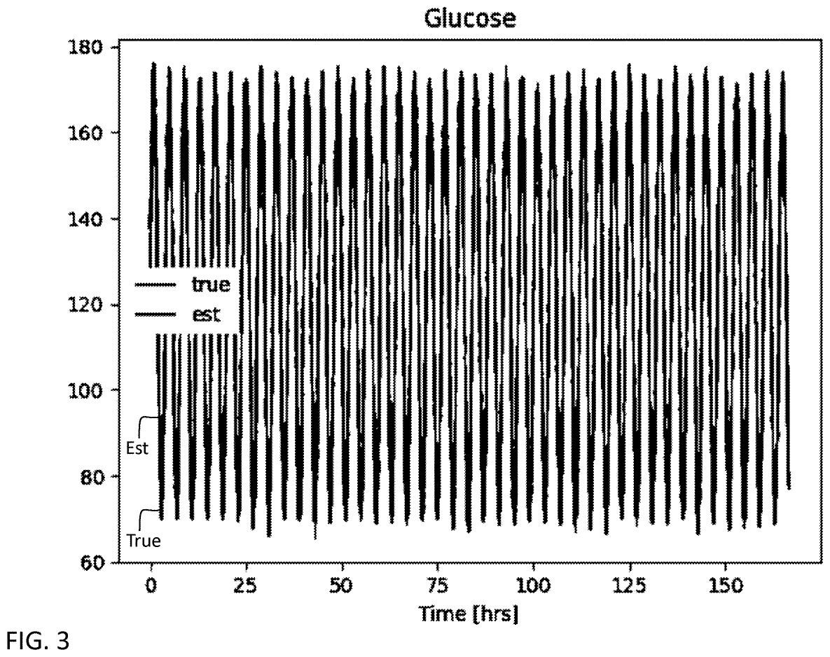

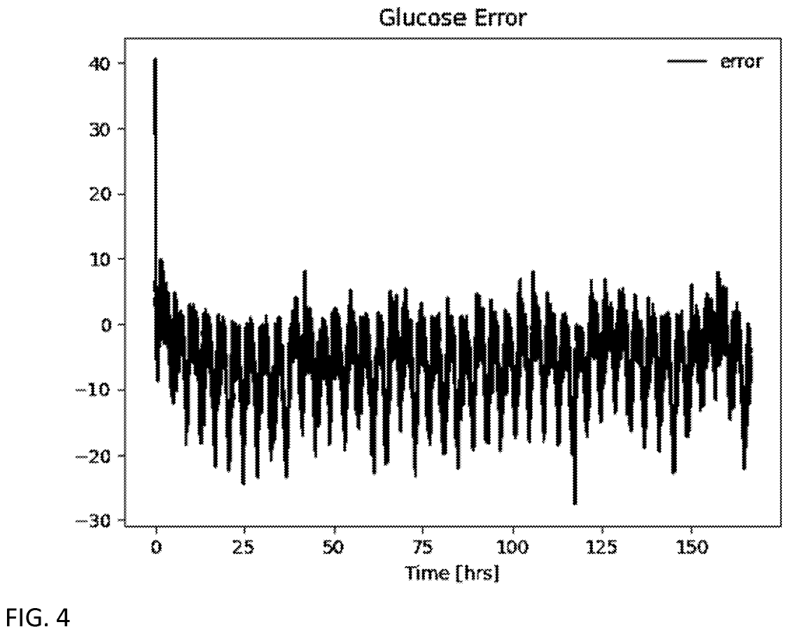

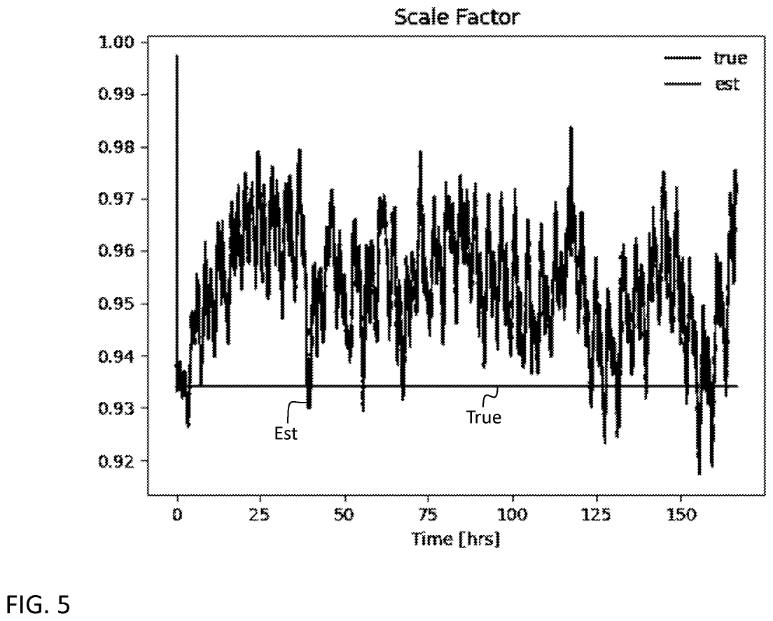

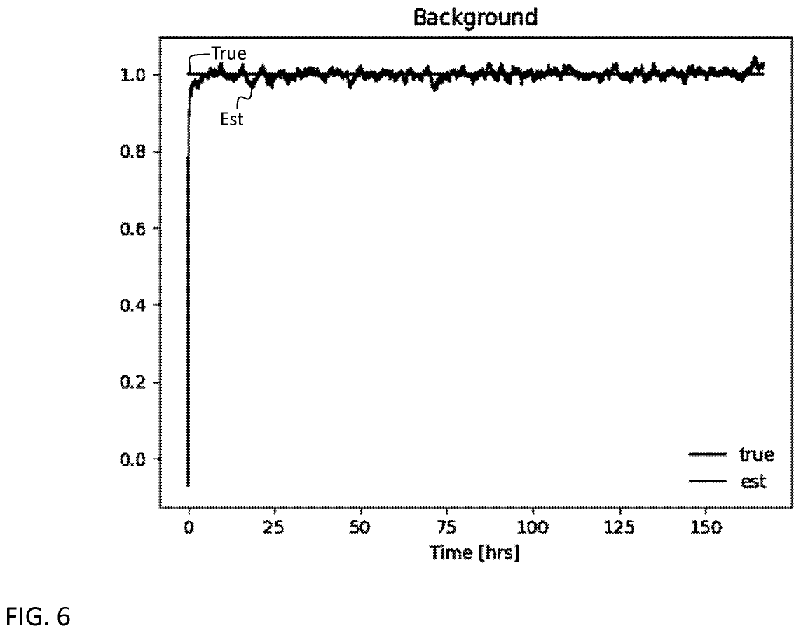

Simulated noisy measurements are shown in FIG. 2, and state vector estimation results from a Monte-Carlo simulation are shown in FIGS. 3-6. FIG. 3 shows the estimated and true glucose concentration as a function of time, FIG. 4 shows the error (the difference between the estimated and true glucose concentrations), FIG. 5 shows the estimated and true scale factor, and FIG. 6 shows the estimated and true background current. Excellent glucose estimation is observed. In FIG. 6, the transient filter response produces negative background current estimates. Clipping the elements of the state vector to expected intervals at each iteration of the filter may eliminate this and potentially accelerate convergence.

Update equations for an Extended Kalman Filter (mentioned above) follow. The prediction step includes state prediction and covariance prediction, with state prediction given by:

x(tₖ/tₖ₋₁)=Fx(tₖ₋₁) (30)
and covariance prediction given by:

P(tₖ|tₖ₋₁)=FP(tₖ₋₁|tₖ₋₁)F^{T}+Q (31)
where F is the time-invariant state transition matrix.

The update step includes the calculation of (i) an innovation, (ii) an innovation covariance, (iii) a Kalman gain, (iv) a state update, and (v) a covariance update.

The innovation may be calculated as follows:

y(tₖ)=z(tₖ)−h(x(tₖ|tₖ₋₁) (32)

The innovation covariance may be calculated as follows:

S(tₖ)=H(tₖ)P(tₖ|tₖ₋₁)H(tₖ)^{T}+R (33)
where

$$H ( {t}_{k} ) = \frac{\partial h}{\partial x} {\vert}_{x ( {t}_{k} \vert {t}_{k - 1} )}$$

is the Jacobian of the nonlinear measurement function h.

The Kalman gain may be calculated as follows:

K(tₖ)=P(tₖ|tₖ₋₁)H(tₖ)^{T}S(tₖ)⁻¹ (34)

The state update may be calculated as follows:

x(tₖ/tₖ)=x(tₖ|tₖ₋₁)+K(tₖ)y(tₖ) (35)

The covariance update may be calculated as follows:

P(tₖ|tₖ)=(I−K(tₖ)H(tₖ)P(tₖ|tₖ₋₁) (36)

As such, as disclosed herein. In some embodiments, a system includes: a first glucose-sensing electrode; and a second glucose-sensing electrode, the first glucose-sensing electrode and the second glucose-sensing electrode being configured to be implanted, separated by less than 5 mm, in a subject, with, e.g., the separation being sufficiently small that the glucose concentration at the two glucose-sensing electrodes is expected to be substantially the same. In some embodiments, the system further includes an electrode carrier, wherein the first glucose-sensing electrode is on the electrode carrier and the second glucose-sensing electrode is on the electrode carrier.

In some embodiments, the first glucose-sensing electrode is separated from the second glucose-sensing electrode by less than 2 mm. In some embodiments: the first glucose-sensing electrode includes a first permeable glucose barrier having a first permeability to glucose, and the second glucose-sensing electrode includes (i) a second permeable glucose barrier having a second permeability to glucose, greater than the first permeability, or (ii) no glucose barrier. In some embodiments, the second glucose-sensing electrode includes a second permeable glucose barrier and the second permeability is at least 1.1 times the first permeability. For example, as discussed herein, differences in the glucose barrier may cause the linearity of the two electrodes to differ, which may be used to estimate the glucose concentration without knowing the in-vitro-to-in-vivo scale factor.

In some embodiments, the system further includes a processing circuit configured to calculate an estimated glucose concentration, the calculation being based on a measured current flowing through the first glucose-sensing electrode and on a measured current flowing through the second glucose-sensing electrode (e.g., it is based on I₀(t) and I₁(t) in Equations 8 through 10). In some embodiments, the calculation is further based on a maximum reaction rate of the first glucose-sensing electrode and a maximum reaction rate of the second glucose-sensing electrode (e.g., it is based on

$${V}_{max}^{( 0 )} and {V}_{max}^{( 1 )}$$

in Equations 8 through 10). In some embodiments, the calculation is further based on a Michaelis constant of the first glucose-sensing electrode and a Michaelis constant of the second glucose-sensing electrode (e.g., it is based on

$${K}_{M}^{( 0 )} and {K}_{M}^{( 1 )}$$

in Equations 8 through 10).

In some embodiments, the estimated glucose concentration is a monotonic function of a true glucose concentration. In some embodiments, the calculating produces an estimated glucose concentration that varies by no more than 5% with a change of 10% in a gain factor common to the first glucose-sensing electrode and the second glucose-sensing electrode. For example, the estimated glucose concentration may be relatively insensitive to the common gain factor (or to the in-vitro-to-in-vivo scale factor), with the estimate changing by only 5% or less if the common gain factor (or the in-vitro-to-in-vivo scale factor) changes by as much as 10%. The use of a glucose estimating function (such as the function of Equation 10) that is insensitive to a common gain factor (e.g., to the in-vitro-to-in-vivo scale factor) may be advantageous if the common gain factor is unknown or not accurately known.

In some embodiments: the calculating includes evaluating a function, the function mapping a first input value x and a second input value y (e.g., I₀(t) and I₁(t), respectively) to an output value z (e.g., ĝ(t)), the function having the property that for x=ky, with k being a constant

$$( e . g . , k being \frac{{I}_{o} ( t )}{{I}_{1} ( t )} ) ,$$

and for x ranging from a first value x0 to a second value x1=1.5 x0, z changes by less than 10%. For example, a set of pairs of electrodes, all implanted in tissue with the same glucose concentration, each set having a common gain constant, the gain constant differing, however, from pair to pair, by as much as a factor of 1.5, would result in substantially the same glucose estimates (e.g., estimates differing by less than 10%) if the ratio

$$\frac{{I}_{0} ( t )}{{I}_{1} ( t )}$$

was the same for each pair.

In some embodiments, the calculating includes evaluating a function, the function mapping a first input value x and a second input value y (e.g., I₀(t) and I₁(t), respectively) to an output value z (e.g., ĝ(t)), the function having the property that for x=ky, for x ranging from a first value x0 to a second value x1=1.5 x0, and for k ranging from a value of 0.7 to a value of 1.3, z is within 10% of (k A−B)/(1−C k) wherein A, B, and C are constants. For example, for

$$k = \frac{{I}_{0} ( t )}{{I}_{1} ( t )} , Equation 10 is \hat{g} ( t ) = \frac{k q {K}_{M}^{( 0 )} - {K}_{M}^{( 1 )}}{1 - k q}$$

$$for q = \frac{{V}_{max}^{( 1 )}}{{V}_{max}^{( 0 )}} ( a constant ) ,$$

corresponding to

$$A = q {K}_{M}^{( 0 )} , B = {K}_{M}^{( 1 )} , and C = q .$$

In some embodiments, A/(BC) is within 10% of a Michaelis constant of the first glucose-sensing electrode divided by a Michaelis constant of the second glucose-sensing electrode

$$( e . g . , for A = q {K}_{M}^{( 0 )} , B = {K}_{M}^{( 1 )} , and C = q , A / ( BC ) = \frac{{K}_{M}^{( 0 )}}{{K}_{M}^{( 1 )}} .$$

In some embodiments, C is within 10% of a maximum reaction rate of the first glucose-sensing electrode divided by a maximum reaction rate of the second glucose-sensing electrode

$$( e . g . , C = \frac{{V}_{max}^{( 1 )}}{{V}_{max}^{( 0 )}} ) .$$

In some embodiments, the calculating includes executing an Extended Kalman Filter. In some embodiments, a state vector of the Extended Kalman Filter includes a glucose concentration (as, e.g., in Equation 20). In some embodiments, a state vector of the Extended Kalman Filter includes a background glucose-sensing electrode current (as, e.g., in Equation 20). In some embodiments, a state vector of the Extended Kalman Filter includes a gain ratio (as, e.g., in Equation 20).

According to an embodiment of the present disclosure, there is provided a method, including: calculating an estimated glucose concentration based on a first sensor signal, from an output of a first glucose-sensing electrode, and a second sensor signal, from an output of a second glucose-sensing electrode, a fit of the current of first glucose-sensing electrode as a function of glucose concentration to a Michaelis-Menten equation resulting in a first Michaelis constant, a fit of the current of second glucose-sensing electrode as a function of glucose concentration to a Michaelis-Menten equation resulting in a second Michaelis constant, differing from the first Michaelis constant by at least 1%.

According to an embodiment of the present disclosure, there is provided a system, including: a first glucose-sensing electrode; and a second glucose-sensing electrode, the first glucose-sensing electrode being implanted in a first tissue space of a subject, the second glucose-sensing electrode being implanted in a second tissue space of the subject, the tissues being selected such that their respective glucose concentrations remain within 5% of each other.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X-Y and the second quantity is at most X+Y. As used herein, when a second number is “within Y %” of a first number, it means that the second number is at least (1−Y/100) times the first number and the second number is at most (1+Y/100) times the first number. As used herein, the word “or” is inclusive, so that, for example, “A or B” means any one of (i) A, (ii) B, and (iii) A and B.

Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Further, the use of “may” when describing embodiments of the inventive concept refers to “one or more embodiments of the present disclosure”. Also, the term “exemplary” is intended to refer to an example or illustration. As used herein, the terms “use,” “using,” and “used” may be considered synonymous with the terms “utilize,” “utilizing,” and “utilized,” respectively.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1-35/100) times 10) and the recited maximum value of 13.5 (i.e., (1+35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

It will be understood that when an element is referred to as being “directly connected” or “directly coupled” to another element, there are no intervening elements present. As used herein, “generally connected” means connected by an electrical path that may contain arbitrary intervening elements, including intervening elements the presence of which qualitatively changes the behavior of the circuit. As used herein, “connected” means (i) “directly connected” or (ii) connected with intervening elements, the intervening elements being ones (e.g., low-value resistors or inductors, or short sections of transmission line) that do not qualitatively affect the behavior of the circuit.

Although exemplary embodiments of a system and method for glucose estimation have been specifically described and illustrated herein, many modifications and variations will be apparent to those skilled in the art. Accordingly, it is to be understood that a system and method for glucose estimation constructed according to principles of this disclosure may be embodied other than as specifically described herein. The invention is also defined in the following claims, and equivalents thereof.

What is claimed is:

1. A system, comprising:
a first glucose-sensing electrode; and
a second glucose-sensing electrode,
the first glucose-sensing electrode and the second glucose-sensing electrode being configured to be implanted, separated by less than 5 mm, in a subject.

2. The system of claim 1, further comprising an electrode carrier,
wherein the first glucose-sensing electrode is on the electrode carrier and the second glucose-sensing electrode is on the electrode carrier.

3. The system of claim 2, wherein the first glucose-sensing electrode is separated from the second glucose-sensing electrode by less than 2 mm.

4. The system of claim 1, wherein:
the first glucose-sensing electrode comprises a first permeable glucose barrier having a first permeability to glucose, and
the second glucose-sensing electrode comprises (i) a second permeable glucose barrier having a second permeability to glucose, greater than the first permeability, or (ii) no glucose barrier.

5. The system of claim 4, wherein the second glucose-sensing electrode comprises a second permeable glucose barrier and the second permeability is at least 1.1 times the first permeability.

6. The system of claim 4, further comprising a processing circuit configured to calculate an estimated glucose concentration, the calculation being based on a measured current flowing through the first glucose-sensing electrode and on a measured current flowing through the second glucose-sensing electrode.

7. The system of claim 6, wherein the calculation is further based on a maximum reaction rate of the first glucose-sensing electrode and a maximum reaction rate of the second glucose-sensing electrode.

8. The system of claim 7, wherein the calculation is further based on a Michaelis constant of the first glucose-sensing electrode and a Michaelis constant of the second glucose-sensing electrode.

9. The system of claim 6, wherein the estimated glucose concentration is a monotonic function of a true glucose concentration.

10. The system of claim 6, wherein the calculating produces an estimated glucose concentration that varies by no more than 5% with a change of 10% in a gain factor common to the first glucose-sensing electrode and the second glucose-sensing electrode.

11. The system of claim 6, wherein:
the calculating comprises evaluating a function,
the function mapping a first input value x and a second input value y to an output value z, the function having the property that for x=k y, with k being a constant, and for x ranging from a first value x0 to a second value x1=1.5 x0, z changes by less than 10%.

12. The system of claim 6, wherein:
the calculating comprises evaluating a function,
the function mapping a first input value x and a second input value y to an output value z,
the function having the property that for x=k y, for x ranging from a first value x0 to a second value x1=1.5 x0, and for k ranging from a value of 0.7 to a value of 1.3, z is within 10% of (k A−B)/(1−C k) wherein A, B, and C are constants.

13. The system of claim 12, wherein A/(BC) is within 10% of a Michaelis constant of the first glucose-sensing electrode divided by a Michaelis constant of the second glucose-sensing electrode.

14. The system of claim 12, wherein C is within 10% of a maximum reaction rate of the first glucose-sensing electrode divided by a maximum reaction rate of the second glucose-sensing electrode.

15. The system of claim 6, wherein the calculating comprises executing an Extended Kalman Filter.

16. The system of claim 15, wherein a state vector of the Extended Kalman Filter includes a glucose concentration.

17. The system of claim 15, wherein a state vector of the Extended Kalman Filter includes a background glucose-sensing electrode current.

18. The system of claim 15, wherein a state vector of the Extended Kalman Filter includes a gain ratio.

19. A method, comprising:
calculating an estimated glucose concentration based on a first sensor signal, from an output of a first glucose-sensing electrode, and a second sensor signal, from an output of a second glucose-sensing electrode,
a fit of the current of first glucose-sensing electrode as a function of glucose concentration to a Michaelis-Menten equation resulting in a first Michaelis constant,
a fit of the current of second glucose-sensing electrode as a function of glucose concentration to a Michaelis-Menten equation resulting in a second Michaelis constant, differing from the first Michaelis constant by at least 1%.

20. The method of claim 19, wherein:
the calculating comprising evaluating a function,
the function mapping a first input value x and a second input value y to an output value z, the function having the property that for x=ky, with k being a constant, and for x ranging from a first value x0 to a second value x1=1.5 x0, z changes by less than 10%.

21. The method of claim 19, wherein the function further has the property that for x=ky, for x ranging from a first value x0 to a second value x1=1.5 x0, and for k ranging from a value of 0.7 to a value of 1.3, z is within 10% of (k A−B)/(1−C k) wherein A, B, and C are constants.

22. The method of claim 21, wherein A/(BC) is within 10% of a Michaelis constant of the first glucose-sensing electrode divided by a Michaelis constant of the second glucose-sensing electrode.

23. The method of claim 21, wherein C is within 10% of a maximum reaction rate of the first glucose-sensing electrode divided by a maximum reaction rate of the second glucose-sensing electrode.

24. The method of claim 19, wherein the calculating comprises executing an Extended Kalman Filter.

25. The method of claim 22, wherein a state vector of the Extended Kalman Filter includes a glucose concentration.

26. The method of claim 22, wherein a state vector of the Extended Kalman Filter includes a background glucose-sensing electrode current.

27. The method of claim 22, wherein a state vector of the Extended Kalman Filter includes a gain ratio.

28. A system, comprising:
a first glucose-sensing electrode; and
a second glucose-sensing electrode,
the first glucose-sensing electrode being implanted in a first tissue space of a subject,
the second glucose-sensing electrode being implanted in a second tissue space of the subject,
the tissues being selected such that their respective glucose concentrations remain within 5% of each other.
